(19) United States
(12) Patent Application Publication
(10) Pub. No.: US 2009/0069674 A1
(43) Pub. Date: Mar. 12, 2009

(54) MEASUREMENT APPARATUS

(75) Inventors: TAKAHIRO MASUMURA, Tokyo (JP); HIROSHI NISHIHARA, Kawasaki-shi (JP); HIROFUMI YOSHIDA, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/203,279

(22) Filed: Sep. 3, 2008

(30) Foreign Application Priority Data
Sep. 12, 2007 (JP) 2007-236711

(51) Int. Cl. A61B 5/00

(52) U.S. Cl. 600/425; 600/476

(57) ABSTRACT

A measurement apparatus is configured to measure a spectroscopic characteristic of a measurement site in a specimen by applying acousto-optical tomography. The measurement apparatus includes a measurement unit configured to measure a light intensity of each of measurement areas that are set differently from the measurement site on a light propagation path from the measurement site to a detection position of a light detector and a signal processing device configured to sequentially modify the spectroscopic characteristics of the measurement areas and the measurement site on the light propagation path from the detection position of the light detector to the measurement site by using a light intensity that is measured by the measurement unit in the measurement area that is closer to a surface layer of the specimen than the measurement site.

### BACKGROUND OF THE INVENTION

1. Field of the Invention

The present invention relates to a measurement apparatus configured to measure a spectroscopic characteristic of a measurement site.

2. Description of the Related Art

A conventional measurement apparatus as used for optical mammography can create an image of a spatial distribution of a spectroscopic characteristic or a metabolism of a biological tissue by observing a spectroscopic characteristic or an attenuation characteristic in the biological tissue. The measurement apparatus creates the image of the spectroscopic characteristic, and needs to measure a biological tissue with a high resolution. The spectroscopic characteristic includes an absorption (spectroscopic) characteristic and a scattering (spectroscopic) characteristic, and acquisitions of both the absorption characteristic and the scattering characteristic (hereinafter referred as “absorption-scattering characteristic”) are necessary to measure the biological tissue with a high resolution. For example, the absorption characteristic of the light enables an amount of each ingredient to be calculated such as hemoglobin, collagen, and water.

Conventional measurement apparatuses apply the Acousto-Optical Tomography (“AOT”) or the Photo-Acoustic Tomography (“PAT”). The AOT irradiates the coherent light and a focused ultrasound into the biological tissue, and detects the modulated light by a light detecting device using an effect of light modulation (an acousto-optical effect) in an ultrasound focusing area (a measurement site), as disclosed in U.S. Pat. No. 6,738,653. On the other hand, the PAT utilizes a difference in absorption factor of the light energy between a measurement site, such as a tumor, and another tissue, and receives through a transducer an elastic wave (an ultrasound or a photoacoustic signal) that occurs as a result of that the measurement site absorbs the irradiated light energy and instantly swells. For example, the PAT is disclosed in U.S. Pat. No. 5,840,023 and A. Oraevsky et al., “Measurement of tissue optical properties by time-resolved detection of laser-induced transient stress,” Appl. Opt., vol. 36, No. 1, pp. 402-415 (1997).

Other prior art include Japanese Patent No. 3,107,914, and S. Feng et al., “Photon migration in the presence of a single defect: a perturbation analysis,” Appl. Opt., Vol. 34, No. 19, pp. 3826-3837 (1995).

In the AOT, the modulated light is absorbed and diffused in a propagation path to the light detecting device, and the light propagation path between the specimen and the light detecting device has a spindle shape. Since the modulated light is affected by the light propagation path, a local spectroscopic characteristic of the measurement area cannot be extracted. U.S. Pat. No. 6,738,653 may provide the metabolism calorie of the entire tissue which spreads like a spindle but cannot provide the metabolism calorie of the measurement site that is a local area in the tissue. In the PAT, the amplitude of the optical signal is proportional to an absorption coefficient in the measurement area. In order to precisely estimate the absorption coefficient of the measurement site, the light intensity of the measurement area needs to be precisely predicted but both U.S. Pat. No. 5,840,023 and “Measurement of tissue optical properties by time-resolved detection of laser-induce transient stress,” supra are silent about an estimation method. It is conceivable, as disclosed in Japanese Patent No. 3,107,914, to use a method of assuming an internal distribution and reconstructing the assumed internal distribution by using an algorithm of changing the assumption based on the measurement result. However, this method requires complex, huge, and time-consuming calculations, and is less likely to converge to an optimal solution quickly.

### SUMMARY OF THE INVENTION

The present invention is directed to a measurement apparatus configured to relatively easily measure a local absorption-scattering characteristic of a specimen with a high precision.

A measurement apparatus according to one aspect of the present invention is configured to measure a spectroscopic characteristic of a measurement site in a specimen by applying acousto-optical tomography. The measurement apparatus includes a measurement unit configured to measure a light intensity of each of measurement areas that are set differently from the measurement site on a light propagation path from the measurement site to a detection position of a light detector and a signal processing device configured to sequentially modify the spectroscopic characteristics of the measurement areas and the measurement site on the light propagation path from the detection position of the light detector to the measurement site by using a light intensity that is measured by the measurement unit in the measurement area that is closer to a surface layer of the specimen than the measurement site.

Further detailed objects and other characteristics of the present invention will become apparent by the preferred embodiments described below referring to accompanying drawings which follow.

### BRIEF DESCRIPTION OF THE DRAWINGS

FIG. 1 is a block diagram of a measurement apparatus according to a first embodiment of the present invention.

FIG. 2 is a schematic cross sectional view of a vessel of the measurement apparatus shown in FIG. 1.

FIG. 3 is a schematic plan view which shows a propagation path of the light that propagates from the measurement site in FIG. 2 to the light detecting device and shows the measurement area located therein.

FIG. 4 is a flowchart which describes an operation of a signal processing device in the measurement apparatus shown in FIG. 1.

FIG. 5 is a schematic sectional view which describes the steps 100 and 101 shown in FIG. 4.

FIG. 6 is a schematic cross sectional view which describes the steps 100 and 101 shown in FIG. 4.

FIG. 7 is a flow chart which describes an operation of a signal processing device in a measurement apparatus according to a second embodiment.

FIG. 8 is a block diagram of a measurement apparatus according to a third embodiment of the present invention.

FIG. 9 is schematic sectional view which shows a relationship between an incident position of the light and the measurement site shown in FIG. 8.

FIG. 10 is a flowchart which describes an operation of the signal detecting unit in the measurement apparatus shown in FIG. 8.

### DESCRIPTION OF THE EMBODIMENTS

Referring now to the accompanying drawings, a description will be given of embodiments of the present invention.

### First Embodiment

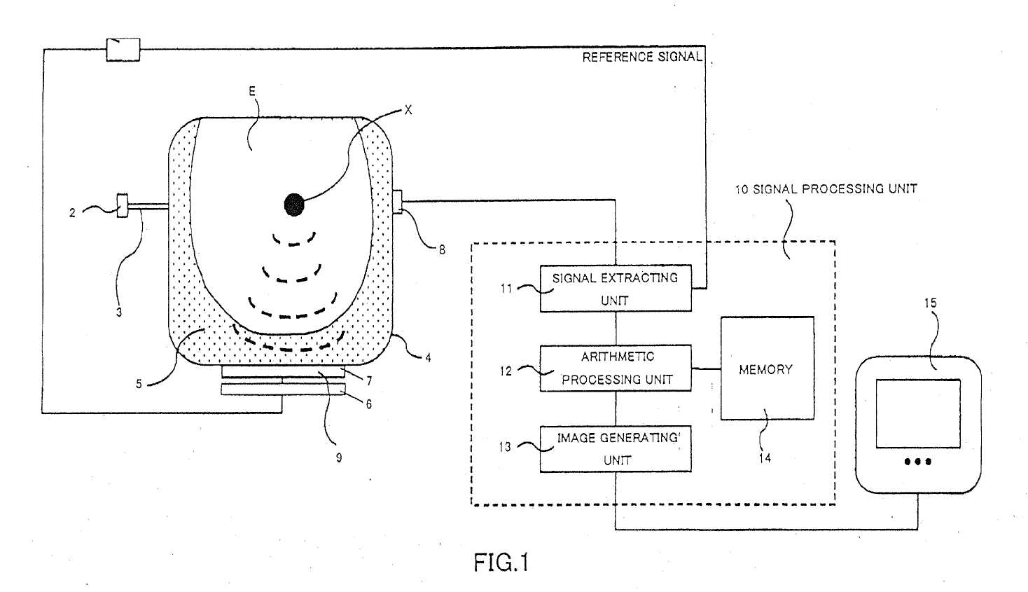

FIG. 1 is a block diagram of an AOT measurement apparatus 100 according to a first embodiment of the present invention. The measurement apparatus 100 is configured to measure an absorption-scattering characteristic in the biological tissue of the specimen E using the AOT, and includes a measurement unit, a signal processing device 10, and a display device 15.

The specimen E has a biological tissue, such as a breast, and also an absorption-scattering body.

The measurement unit includes a sinusoidal oscillator 1, a light source 2, an optical fiber 3, a measurement vessel 4, a matching material 5, an ultrasound oscillator (an ultrasound transducer array) 6, an ultrasound focusing device 7, and a light detector (detecting device) 8.

The sinusoidal oscillator 1 drives the ultrasound generating device 6 at a sinusoidal signal of a frequency f.

The light source 2 is a light source configured to generate the luminous fluxes having a plurality of wavelengths to be irradiated on the specimen E. A wavelength of the light source is selected among wavelengths in accordance with absorption spectra of water, lipid, protein, oxygenated hemoglobin, and deoxygenated hemoglobin. In an example, an appropriate wavelength falls upon a range between 600 to 1500 nm, because that light can highly transmit due to a small absorption of water that is a main ingredient of the internal biological tissue, and provides a characteristic spectrum for lipid, oxygenated hemoglobin, and deoxygenated hemoglobin. The laser source has a long coherence length, such as 1 m or greater, and generates continuous wave (“CW”) light having a constant intensity. The laser source may apply a semiconductor laser or a wavelength-variable laser that can generate various different wavelengths.

The optical fiber 3 guides the light emitted from the light source 2 to the specimen E. A light collecting (condenser) optical system that can efficiently guide the light from the light source 2 to the end of the optical fiber 3 may be provided prior to the optical fiber 3. The light which enters the measurement vessel 4 propagates while repeating absorptions and scatters.

The measurement vessel 4 houses the specimen E and the matching material 5. The measurement vessel 4 is made of a material that transmits a wavelength of the light emitted from the light source 2. The matching material 5 is made of an acoustic impedance material that efficiently transmits the ultrasound to the specimen E. The matching material 5 is filled in a space between the specimen E and the measurement vessel 4. A refractive index, an absorption coefficient, a scattering coefficient, and an acoustic characteristic of the matching material 5 are already known.

The ultrasound generating device 6 generates an ultrasound (pulse). The ultrasonic frequency ranges from 1 to several tens (of MHz) although it may vary with a measurement depth of the specimen E or a resolution. For example, the ultrasound generating device 6 includes a linear array search unit. This embodiment uses an ultrasonic transducer array in which an ultrasonic oscillator is integrated with an ultrasound detecting device.

The ultrasound focusing device 7 focuses an ultrasound emitted from the ultrasound generating device 6 onto the measurement site (the ultrasound focusing area) X in the tissue of the specimen E. A method of focusing the ultrasound includes a method of using a circular concave ultrasonic transducer or an acoustic lens, or an electric focusing method that uses an array search unit. At the measurement site X, a sound pressure changes a density of the medium, causing a change in a refractive index of the medium and a displacement of the scatters. When the light passes through the measurement site X, a phase of the light is modulated with the ultrasonic frequency f due to the change of the refractive index of the medium and the displacement of the scatters. This phenomenon will be referred as an acousto-optical effect.

The light detector 8 detects the light that has been modulated by the acousto-optical effect at the measurement site X of the specimen E. The light detector 8 may apply a photoelectric conversion device such as a photomultiplier tube (“PMT”), a charge coupled device (“CCD”), and a complementary metal-oxide semiconductor (“CMOS”). The light detector 8 when using the PMT, for example, can detect a signal from both the modulated light and the non-modulated light. A signal extracting unit 11 in the signal processing device 10 Fourier-transforms the detected signal, and separates the non-modulated signal I1 from the modulated signal I2. The non-modulated signal I1 and the modulated signal I2 are used to calculate the spectroscopic characteristic of the specimen E as described in U.S. Pat. No. 6,738,653.

The signal processing device 10 generates an image of the spectroscopic characteristic at the measurement site of the specimen E, and includes the signal extracting unit 11, a processing unit 12, an image generating unit 13, and a memory 14. The signal extracting unit 11 serves as a filter, and separates the modulated light from the non-modulated light. The signal extracting unit 11 may apply a band pass filter which selectively detects a signal having a specific frequency and a lock-in amplifier which amplifies and detects the light having a specific frequency. The processing unit 12 calculates a concentration and a constituent ratio of an ingredient that contributes to the spectroscopic characteristic or the absorption of the spectroscopic characteristics. The processing unit 12 generates distribution data for the spectroscopic characteristic in the specimen E based on coordinate data of the focused ultrasound and an optical signal corresponding to the coordinate data. At this time, the processing unit 12 modifies a measurement result of the measurement unit as described later. The image generating unit 13 generates a three-dimensional tomographic image (or image) of the specimen E based on the distribution data of the spectroscopic characteristic in the specimen E generated by the processing unit 12. The memory 14 records data generated by the processing unit 12, and an image of the spectroscopic characteristic generated by the image generating unit 13. The memory 14 may use a data storage, such as an optical disc, a magnetic disc, a semiconductor memory, and a hard disk drive.

The display device 15 displays an image generated by the signal processing unit 10, and can use as a liquid crystal display, a CRT, or an organic EL.

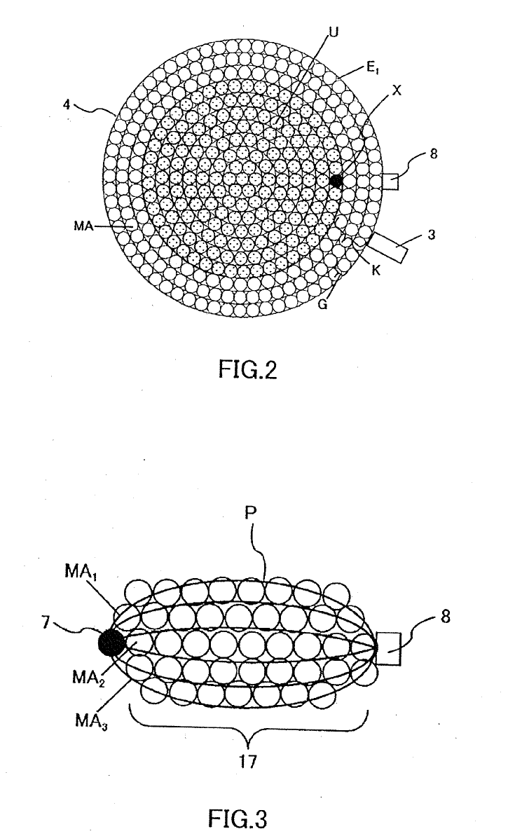

FIG. 2 is a schematic sectional view of the measurement vessel 4. For simplification, FIG. 2 shows the measurement vessel 4 filled with the specimen E on a certain section. A surface layer E₁ which is an outer surface of the specimen E accords with the outer surface of the measurement vessel 4. Of course, the matching material 5 may be arranged between the specimen E and the measurement vessel 4.

K in FIG. 2 denotes an area in which the absorption-scattering characteristic has already been known or measured. U denotes an area on which absorption-scattering characteristic has not yet been known or measured. G is an annular outermost area closest to the surface layer E₁ of the specimen E. MA denotes a measurement area concentrically arranged in the specimen E, and may include the measurement site X having a target spectroscopic characteristic is to be measured. The measurement site X is located in the circular area U. The measurement site having a spectroscopic characteristic to be measured may be the entire measurement area MA. The measurement site X is not necessarily distinguished from the measurement area MA. The area K is arranged between the area U and the surface layer E₁ of the specimen E. This embodiment recursively calculates the spectroscopic characteristics of the measurement site X and the measurement area MA using the spectroscopic characteristic of the outermost area G. The present invention allows a recursive calculation of at least one of the absorption characteristic and the scattering characteristic of the measurement site X by using that corresponding to at least one of the outermost area. Here, the spectroscopic characteristics of the measurement site X and the measurement area MA are recursively calculated by using the spectroscopic characteristic of the outermost area (an area which is closest to the surface layer of the specimen) G which has a circular and coronal shape, but the calculation is not limited to this embodiment. The measurement site X and the measurement area MA can be also calculated based on the spectroscopic characteristics (measurement results) of an area closer to the surface layer of the specimen than the measurement site X and the measurement site MA (an area in which the spectroscopic characteristic can be measured more precisely than in the measurement site x and the measurement area MA).

As shown in FIG. 2, this embodiment sets the measurement areas MA over the entire area inside the measurement vessel 4, and calculates their spectroscopic characteristic from the external spectroscopic characteristic. The present invention does not limit an arrangement of the measurement areas MA, although FIG. 2 concentrically arranges the measurement areas MA. As shown in FIG. 3, the measurement areas MA can be set on the light propagation path from the measurement site X to the light detector 8. This embodiment uses a difference between an actual measurement value and a predicted value of the light intensity at the measurement site X, and calculates the predicted value by using the measurement result of the measurement area MA outside the measurement site X, as described later.

FIG. 3 is a schematic plan view of the light propagation path P between the measurement site X and the light detector 8, and the measurement areas MA arranged thereon. A spectroscopic characteristic of each measurement area MA on the light propagation path P between the measurement site X and the light detector 8 belongs to the interior of the known area K. At this time, a light incident position of the optical fiber 3 and a detection position of the light detector 8 are set such that the light detector 8 can measure the light that is introduced from the light incident position, and then reflected on the measurement areas MA or the measurement site X. The light incident position of the optical fiber 3 and the detection position of the light detector 8 are configured movable. As a result, they constitute a relationship of a reflection type measurement system which mainly measures the backward scattering light, as shown in FIG. 2. Thereby, the light incident position and the detection positions can be set such that all paths to the detection position of the light detector 8 via the measurement site X can exist in the area K. As a result, the spectroscopic characteristic of only the measurement site X can be measured without influence of the area K. The spectroscopic characteristic of the measurement site X which is a local area tagged by the acousto-optical effect can be recursively calculated from the area K having a known spectroscopic characteristic.

In measuring a spectroscopic characteristic of the yet-measured area U, the spectroscopic characteristic of the measurement site X is obtained by calculating a difference of the light intensity between the actual measurement value of the light intensity and the light intensity that is obtained from a measurement result of the area K and by eliminating the influence of the area K. This flow is repeated, and the spectroscopic characteristic of the measurement area MA on the path can be recursively calculated from the outermost area G. By mapping the absorption characteristic and the scattering characteristic with the position of the measurement site X, a tomographic image of one section of the specimen E can be obtained. The three-dimensional absorption-scattering information on the specimen E can be ultimately obtained by scanning this section.

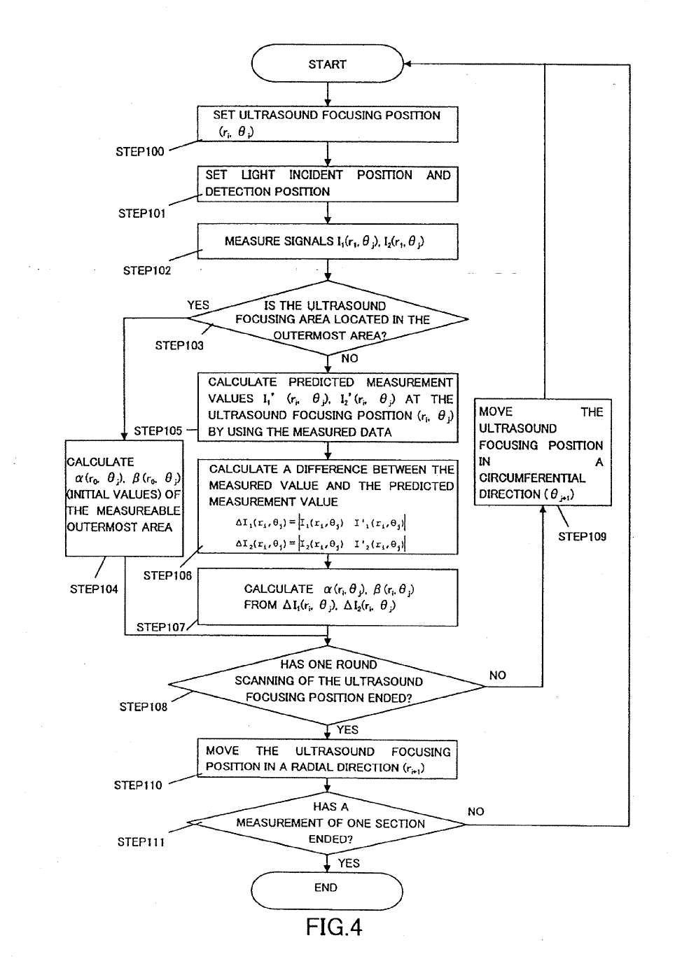

FIG. 4 is a flowchart for explaining an operation of the signal processing device 10 (or the processing unit 12) in obtaining the tomographic image of one section of the specimen E.

Initially, the step 100 sets the measurement area MA as an ultrasound focusing position. This position may be determined by controlling the ultrasound focusing device 7. Next, the step 101 adjusts the light incident position of the optical fiber 3 and the detection position of the light detector 8 so as to form the reflection type measurement, and sets an interval between them such that an average distribution of the light propagation path P can fall upon the area K. The processing part 12 calculates the light propagation path P by using the diffusion theory or the Monte Carlo method and the absorption-scattering characteristic that has been already measured. The light incident position and the detection position can be properly varied depending upon a position of the measurement site X.

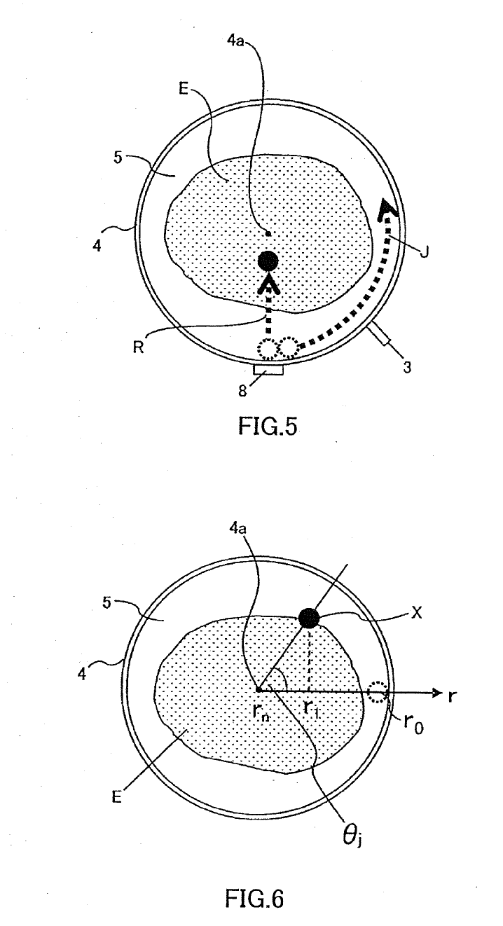

The light detecting device 8 is arranged adjacent to the side surface of the measurement vessel 4 on an extension from the center 4a of the measurement vessel 4 to the measurement site X. Assume a radial coordinate rᵢ (i=0 to n) from the boarder of the measurement vessel 4 to the center 4a and a circumferential deviation angle θⱼ (j=0 to m) as shown in FIG. 6, in a two-dimensional polar coordinate system with the center 4a as an origin on one section of the measurement vessel 4. The number of divisions of θⱼ depends upon the position rᵢ.

The step 102 measures the non-modulated light's intensity I₁(r₁,θⱼ) and the modulated light's intensity I₂(r₁,θⱼ) at a position (r₁,θⱼ) of the measurement site X or the measurement area MA. This embodiment first sets one measurement area MA near the boundary in the section of the measurement vessel 4 as shown in FIG. 5, and sequentially and adjacently shifts a position of the measurement area MA in the circumferential direction J for each measurement. The step 102 initially measures the non-modulated light's intensity I₁(r₀,θⱼ) and modulated light's intensity I₂(r₀,θⱼ). The non-modulated light's intensity I₁(r₀,θⱼ) and modulated light's intensity I₂(r₀,θⱼ) in the outermost area (i.g., r₀) are measurable directly rather than recursively.

The step 103 determines whether a position r₀ that is an outer circumference near the boundary of the measurement vessel 4 has been measured for the measurement site X. In measuring the outermost area, the method described in U.S. Pat. No. 6,738,653 is, for example, used to calculate the absorption characteristic α(r₀,θⱼ) and the scattering characteristic β(r₀,θⱼ) (step 104). The absorption characteristic α(r₀,θⱼ) is an attenuation coefficient of the intensity by absorptions, and the scattering characteristic β(r₀,θⱼ) is an attenuation coefficient of the intensity by scatters. In FIG. 5, this embodiment moves a position of the measurement area MA to the inside by one along a radial direction R after one round measurement ends, so as to repeat a similar measurement. Thus, the step 109 moves a position of the measurement site X to a position that is adjacent to the present position in the circumferential direction until one round measurement ends at the position r₀ in the step 108.

The step 100 sets an ultrasound focusing position, repeats a measurement, and calculates the absorption characteristic α(r₀,θⱼ) and the scattering characteristic β(r₀,θⱼ) of the outermost area in the measurement vessel (step 104). The memory 14 in the signal processing device 10 sequentially records measurement data that is measured at the position (r₀,θⱼ) of the measurement area MA and a calculated absorption-scattering characteristic. After one round measurement of the outermost area ends in the step 108, the step 110 moves the measurement area MA to the inside by one along the radial direction R. The step 111 returns to the step 100, and the step 102 measures the non-modulated light intensity I₁(rᵢ,θⱼ) and the modulated light intensity I₂(rᵢ,θⱼ)

The flow moves to the step 105 from the step 103. The step 105 calculates predicted values I′₁(rᵢ,θⱼ) and I′₂(rᵢ,θⱼ) of the non-modulated light and the modulated light to be measured by the light detector 8 under the current measurement condition by utilizing a measurement result of the step 104.

The predicted values I′₁(r₁,θⱼ) and I′₂(r_{1,θ}ⱼ) can be expressed as follows by the non-modulated light intensity I₁(r₀,θₖ) and the modulated light intensity I₂(r₀,θₖ) that are known or actual measurement values:

I₁′(r₁,θⱼ)=β(r₁,θⱼ)I₁(rₒ,θₖ)exp └−α(r₁,θⱼ)L┘

I₂′(r₁,θⱼ)=β(r₁,θⱼ)I₂(rₒ,θₖ)exp [−α(r₁,θⱼ)L] EQUATION 1

L is a diameter of the measurement area MA.

Equation 1 is expandable to r=1 and r=i−1 (i is 2 or greater), as given by the following equation:

I₁′(rᵢ,θⱼ)=β(rᵢ,θⱼ)I₁(rᵢ₋₁,θₖ)exp └−α(rᵢ,θⱼ)L┘

I₁′(rᵢ,θⱼ)=β(rᵢ,θⱼ)I₁(rᵢ₋₁,θₖ)exp [−α(rᵢ,θⱼ)L] EQUATION 2

A light intensity of a new measurement area MA or the measurement site X at the position of r=i and θ=j is predicted by the light intensities of the measurement areas MA on the light propagation paths among the measurement areas at positions of r=i−1. For example, the light intensity of the measurement site X in FIG. 3 is predicted by the light intensities of three right adjacent measurement areas MA₁ to MA₃. θₖ defines this range, which is a banana-shaped optical path distribution determined by the absorption-scattering characteristic of the medium and a distance between the light source and the detecting device, as described in “Photon migration in the presence of a single defect: a perturbation analysis,” supra.

Other than the above method, the optical diffusion equation may be solved, for example, by a finite element method using the absorption characteristic α(rᵢ,θⱼ) and the scattering characteristic β(rᵢ,θⱼ) of the measurement area MA in the area K shown in FIG. 2, which has the known absorption-scattering characteristic, or the predicted values I′₁(rᵢ,θⱼ) and I′₂(rᵢ,θⱼ) of the non-modulated light and the modulated light to be measured by the light detector 8 may be directly calculated, for example, by using the Monte Carlo simulation.

The step 106 calculates differences ΔI₁(rᵢ,θⱼ) and ΔI₂(rᵢ,θⱼ) between the measured values and the predicted values. The differences also may be obtained by interpolating a plurality of adjacent measurement points when there are no measurement points having the same deviation angle θⱼ.

ΔI₁(rᵢ,θⱼ)=|I₁(rᵢ,θⱼ)−I′₁(rᵢ,θⱼ)|

ΔI₂(rᵢ,θⱼ)=|I₂(rᵢ,θⱼ)−I′₂(rᵢ,θⱼ)| EQUATION 3

Based on the measurement result obtained from Equation 3 in the step 107, α(rᵢ,θⱼ) and β(rᵢ,θⱼ) are calculated when the ultrasound focusing position is located at (rᵢ,θⱼ). Here, assume the absorption-scattering characteristic of the measurement area MA or the measurement site X at the position (rᵢ,θⱼ) by the following equation, although it may also be obtained by interpolating adjacent measurement points when there are no measurement points having the same deviation angle θⱼ.

α(rᵢ,θⱼ)=α(rᵢ₋₁,θⱼ)

β(rᵢ,θⱼ)=β(rᵢ₋₁,θⱼ) EQUATION 4

Based on the differences derived from Equation 3, deviation amounts δα(rᵢ,θⱼ) and δβ(rᵢ,θⱼ) from the absorption-scattering characteristic on the assumption of Equation 4 are set by the following equation, and the equation 4 is modified.

α(rᵢ,θⱼ)=α(rᵢ₋₁,θⱼ)+δα(α(rᵢ,θⱼ))

β(rᵢ,θⱼ)=β(rᵢ₋₁,θⱼ)+δβ(α(rᵢ,θⱼ)) EQUATION 5

A local absorption-scattering characteristic in the area of the measurement site X can be obtained by eliminating the influence that propagates the area K through a differencing process. In other words, as indicated by Equation 4, the processing unit 12 assumes that two adjacent measurement areas have the same spectroscopic characteristic on the light propagation path P. Next, the processing unit 12 obtains a difference ΔI between an actual measurement value I of the light intensity of one of two adjacent measurement areas which one is closer to the measurement site than the other measurement area, and a predicted value I′ of the light intensity of the one measurement area predicted based on a measurement result of the other measurement area of the two adjacent measurement areas which is closer to the light detecting device than the one measurement area. Then, the processing unit 12 modifies the spectroscopic characteristic on the one measurement area as in Equation 5 based on a deviation amount δ which corresponds to this difference.

The above flow is repeated in the step 108 until the measurement of one round ends at the position rᵢ. Whenever the one round measurement ends, the step 110 moves the measurement area MA to the inside along the radial direction R, and performs the similar process. This flow is repeated to continue the measurements to the center 4a in the measurement vessel 4. The flow shown in FIG. 4 can provide the attenuation coefficient α(rᵢ,θⱼ) related to local absorptions and the attenuation coefficient β(rᵢ,θⱼ) related to local scatters on a section including the specimen E and the matching material 5.

Thus, the processing unit 12 modifies the measurement result of the measurement site X in the specimen E measured by the measurement unit. In modification, the processing unit 12 uses the light intensity of the measurement area MA in the outermost area measured by the measurement unit, and modifies a spectroscopic characteristic of the measurement area MA on the light propagation path in a direction W shown in FIG. 3 from the light detector 8 to the measurement site X. Then, the processing unit 12 modifies the spectroscopic characteristic of the measurement site X measured by the measurement unit based on the modified spectroscopic characteristics of all adjacent measurement areas (such as the measurement areas MA₁ to MA₃ in FIG. 3) on the light propagation path of the measurement unit X.

The image generating unit 13 may obtain a tomographic image of the absorption-scattering characteristic in the specimen E by mapping α(rᵢ,θⱼ), β(rᵢ,θⱼ) at the position (rᵢ,θⱼ). The above flow allows the display device 15 to display the spectroscopic characteristic by modifying the spectroscopic characteristic and measuring it on a real time basis.

The absorption characteristic α(rᵢ,θⱼ) at each position (rᵢ,θⱼ) is measured with a plurality of wavelengths, and the Beer Lambert Law is applied to the area of the measurement site X. A constituent of the main ingredient of the specimen E can also be analyzed by fitting a weight by the absorption characteristic of that ingredient. For example, a concentration or ratio of a main organic ingredient, such as oxygenated hemoglobin, deoxygenated hemoglobin, water, lipid, and collagen, is calculated, and its distribution in the organism is displayed as a tomographic image. Alternatively, from a ratio between oxygenated hemoglobin and deoxygenated hemoglobin, a metabolic image such as the oxygen saturation of hemoglobin may be visualized as a tomographic image.

This embodiment arranges the measurement areas MA in the entire area on one tomographic surface without distinguishing the specimen E from the matching material 5, but may set the measurement areas MA only in the interior of the specimen E and obtain the tomographic image. For example, a boundary between the specimen E and the matching material 5 is measured based on an echo signal from the ultrasound generating device 6. The measurement site X is set adjacent to or inside of the boundary, and the measurement of the step 102 is implemented. On the other hand, in the calculation of a difference value in the step 106, boundary areas α(r₀,θⱼ) and βα(r₀,θⱼ) of the specimen E may be calculated by using the matching material 5 having the known absorption-scattering characteristic. A flow similar to that of FIG. 4 and the measurement result are used to calculate the absorption-scattering characteristic of the interior of the specimen. This embodiment provides the matching material 5 between the specimen E and the measurement vessel 4, but may directly measure the specimen E without using the matching material 5.

This embodiment first measures the outer circumference of the measurement vessel, and then moves the measurements to the inside concentrically, as shown in FIG. 5, but may measure from the outer circumference to the center as long as the flow in FIG. 4 can be established, change a deviation angle, and repeat the measurements from the outer circumference to the center.

### Second Embodiment

The second embodiment also uses the measurement apparatus 100 shown in FIG. 1. The first embodiment measures and calculates the absorption-scattering characteristic on a real-time basis. On the other hand, the second embodiment measures and obtains the measurement data, and then the signal processing device 10 calculates an absorption-scattering characteristic. The second embodiment measures similarly to the first embodiment, but does not limit the measurement order, as long as the measurement areas MA and the measurement site X are set in the entire area as shown in FIG. 2 and their measurement values exist. The memory 14 stores the light intensities of all measurement areas MA measured by the measurement unit before the processing unit 12 starts processing.

In measurement, the memory 14 stores the non-modulated light's intensity I₁ (rᵢ,θⱼ) and the modulated light's intensity I₂(rᵢ,θⱼ) measured at the position (rᵢ,θⱼ) of the measurement site X, and the measurement condition including an arrangement between the optical fiber 3 and the light detector 8. In analyzing data, the processing device 12 sequentially reads the data stored in the memory 14 and analyzes it. This embodiment reads the data from the memory 14 in the same order as the measurement order in the first embodiment.

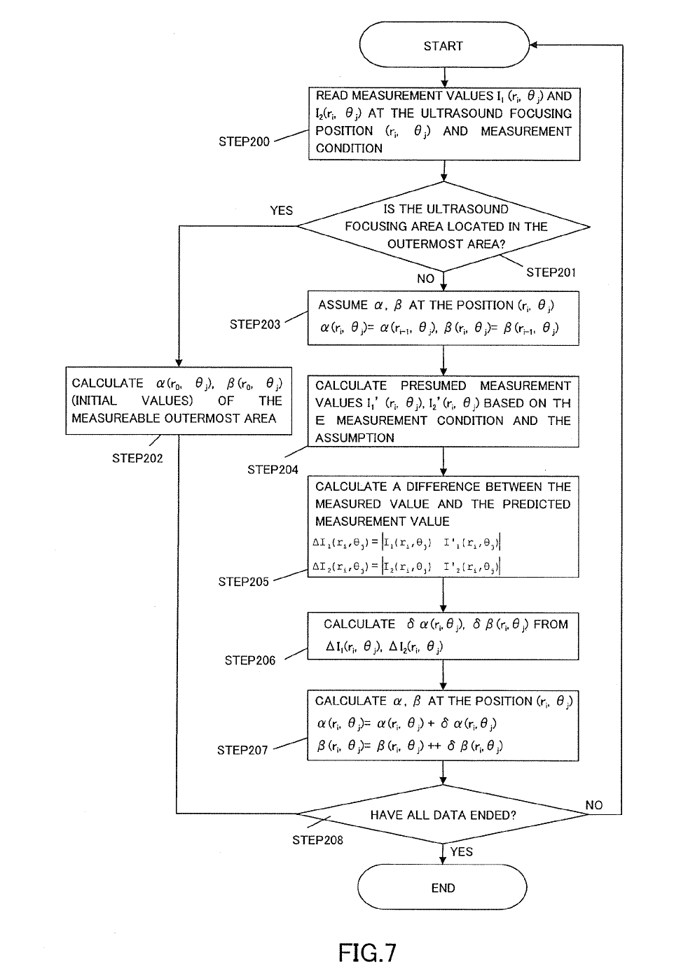

FIG. 7 is a flowchart for explaining an operation of the signal processing device 10 (or the processing unit 12) of this embodiment in obtaining a tomographic image on one section of the specimen E.

The step 200 reads out of the memory 14 the non-modulated light's intensity I₁(rᵢ,θⱼ) and the modulated light's intensity I₂(rᵢ,θⱼ) that are measured at the position (rᵢ,θⱼ) of the measurement site X, and the measurement condition. The data of the outermost area G in the measurement vessel 4 is read out, and the flow moves to the step 202 from the step 201. The step 202 calculates α(r₀,θⱼ) and β(r₀,θⱼ) similarly to the first embodiment. The steps 200 to 202 are repeated via the step 208 in order to calculate the absorption-scattering characteristic of the outermost area G in the measurement vessel 4. Next, data measured in the area adjacent to the measurement site X is read out in the circumferential direction, and the step 201 moves to the step 203. The step 203 assumes Equation 4.

On the assumption of Equation 4, the step 204 calculates photon propagations from the light incident point, the position (rᵢ,θⱼ) of the measurement site X, and the light detector 8. Based on this calculation, the step 204 obtains predicted measurement values I′₁(rᵢ,θⱼ) and I′₂(rᵢ,θⱼ) of the non-modulated light and the modulated light to be measured by the light detector 8, using the optical diffusion equation or the Monte Carlo Simulation.

The step 205 calculates a difference between the measurement value read by the step 200 and the predicted measurement value calculated by the step 204 as in Equation 3. The step 206 calculates deviation amounts δα(rᵢ,θⱼ) and δβ(rᵢ,θⱼ) from the absorption-scattering characteristic on the assumption of Equation 4 based on the calculated difference between the measurement value and the predicted value. The step 207 modifies the deviation amount calculated by the step 206 as in Equation 5, and calculates the absorption-scattering characteristic at the position (rᵢ,θⱼ). The step 208 then reads out all of the measurement data and repeats the flow until the analysis ends.

The flow shown in FIG. 7 can also provide a calculation of the absorption-scattering characteristic according to the position (rᵢ,θⱼ) inside the measurement vessel 4. By mapping the absorption-scattering characteristic with a position coordinate, similar to the first embodiment, a distribution of the absorption-scattering characteristics in the specimen can be easily obtained as a tomographic image. Additionally, each tomographic image may be generated and visualized for each main ingredient by separating it based on a constituent ratio of the main ingredients in the absorption characteristic. Even in this embodiment, the specimen E may be directly measured instead of arranging the matching material 5 between the specimen E and the measurement vessel 4.

### Third Embodiment

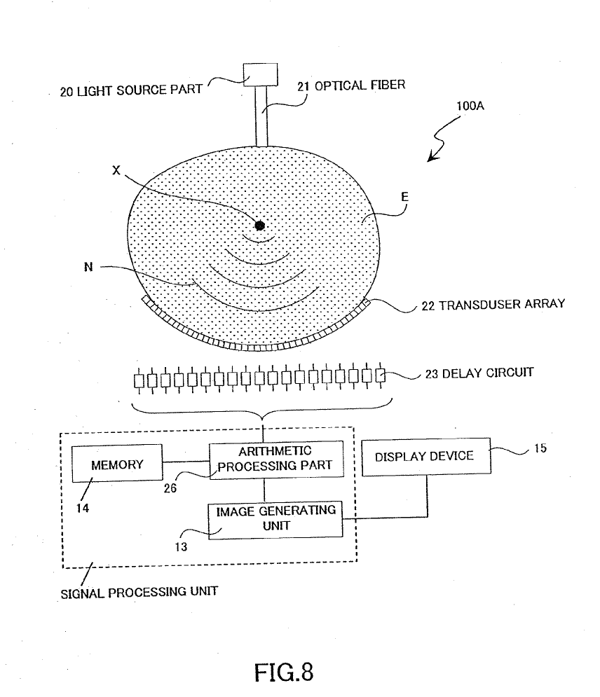

FIG. 8 is a block diagram of a PAT measurement apparatus 100A according to the third embodiment of the present invention. The measurement apparatus 100A uses the PAT to measure the spectroscopic characteristic (the absorption characteristic and the scattering characteristic) in the tissue of the specimen E, and includes the measurement unit, the light detector 8, a delay circuit 23, a signal processing device 24, a processing unit 26, the memory 14, and the display device 15. Those elements in FIG. 8, which are the corresponding elements in FIG. 1, will be designated by the same reference numerals and a description thereof will be omitted. The measurement unit has a light source 20, an optical fiber 21, and an ultrasound detecting device (an ultrasonic transducer array) 22.

The pulsed light is emitted from the light source 20, and enters the specimen E via the optical fiber 21. The energy absorbed in the specimen E is transformed into heat, and induces an elastic wave N through the thermoelastic process. At this time, a pulse width of the light source 20 is set to satisfy a stress confinement condition or narrower than the stress relaxation time. The ultrasound detecting device 22 detects the elastic wave N that is emitted in the specimen E in response to the irradiation of the pulsed light. A focusing area has been previously set, and the delay circuit 23 operates in accordance with the setting and detects a sound pressure from the local measurement site X. The detected signal is transmitted to the signal processing unit 24. As disclosed in “Measurement of tissue optical properties by time-resolved detection of laser-induced transient stress,” supra, the absorption characteristic, the scattering characteristic, and an effective attenuation characteristic of the light can be calculated from the measured sound pressure.

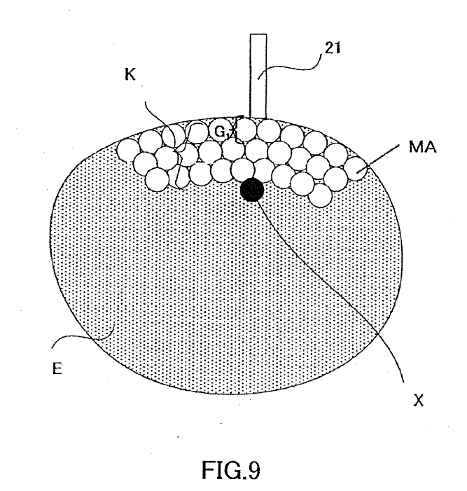

This embodiment also sets the measurement areas in the outermost area near the surface layer in the specimen, and measures them. As shown in FIG. 9, an attenuation amount of the light can be estimated since the light propagation to the measurement site X occurs in the area K having the known absorption-scattering characteristic in the previous measurements. Therefore, the light intensity at the measurement site X may be precisely presumed, and the spectroscopic characteristic of the local measurement site X can be calculated from the light intensity and the measured sound pressure. The present invention that applies the PAT thus can precisely estimate the light intensity of the measurement site X located in the area U by using the spectroscopic characteristic of the area K. This recursive measurement provides the internal distribution of the spectroscopic characteristic through local measurements to the entire area in the specimen.

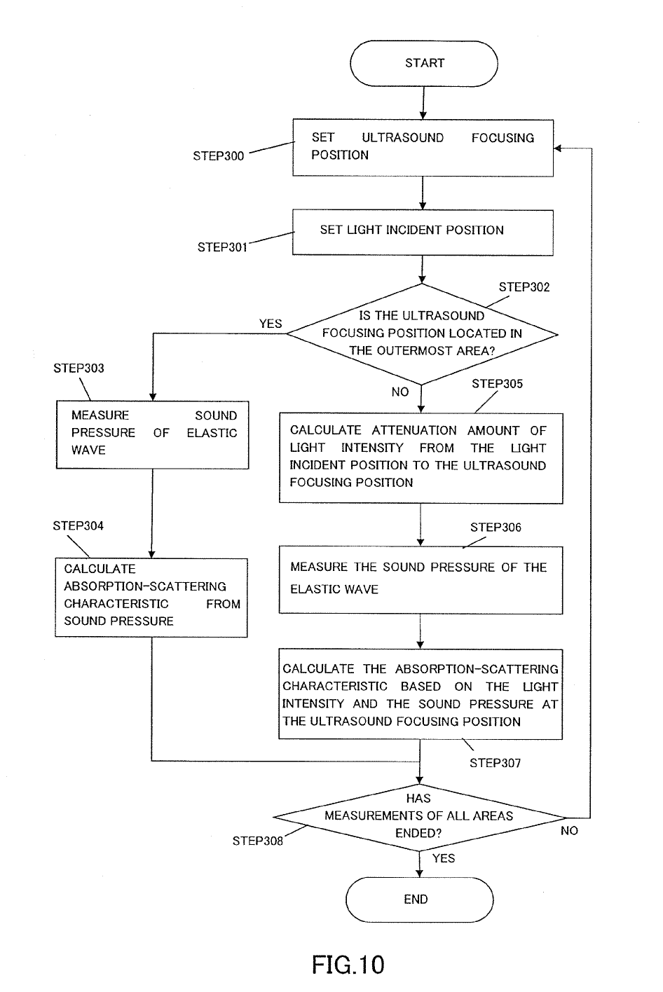

FIG. 10 is a flowchart for explaining an operation of the signal processing device 24 (or the processing unit 26).

Initially, the step 300 sets the measurement site X and the measurement area MA. Next, the step 301 sets an incident position from which the light is incident upon the specimen E so as to make short a distance from the surface of the specimen E to the measurement site X. Next, when the measurement area MA is the outermost area G, the step 302 moves to the step 303 which measures the sound pressure by irradiating the light and detecting the elastic wave N through the ultrasound detecting unit 22. The step 304 calculates a spectroscopic characteristic from the obtained sound pressure by using the following method. The memory 14 sequentially stores the measurement result.

A fluence rate Φ(r,t) of a photon as a light intensity is given by the following equation where r is a position in the absorption-scattering medium, and t is time.

$$\frac{\partial Φ ( r , t )}{\partial t} = D {∇}^{2} Φ ( r , t ) - v {μ}_{a} Φ ( r , t ) + vS ( r , t ) \qquad EQUATION 6$$

Φ(r,t) is a fluence rate of a photon [number of photons/(mm²·sec)]. D(=v/3 μ′ₛ) is a diffusion coefficient [mm²/sec]. μ′ₛ is a reduced scattering coefficient [1/mm]. v is the light speed in the specimen [mm/sec]. μₐ is an absorption coefficient [1/mm]. S(r,t) is irradiation photon flux density of the light source [number of photons/ (mm³·sec)].

In general, a pressure P (r) of the elastic wave at the position r in the absorption-scattering medium is given by the following equation.

$$P ( r ) = \frac{1}{2} {Γμ}_{a} ( r ) Φ ( r ) \qquad EQUATION 7$$

Γ is Gruneisen coefficient (heat—acoustic conversion efficiency). μₐ(r) is an absorption coefficient at the position r. Φ(r) is a fluence rate of a photon at the position r.

The step 304 assumes an absorption coefficient μₐ and a reduced scattering coefficient μ′ₛ of the measurement site X, and uses the Monte Carlo simulation to obtain the light intensity and to calculate a predicted value of the sound pressure. The calculation is repeated to presume the absorption coefficient μₐ and the reduced scattering coefficient μ′ₛ so that the signal predicted value matches the measurement value. The optical diffusion equation may be used instead of the Monte Carlo simulation.

Alternatively, as described in “Measurement of tissue optical properties by time-resolved detection of laser-induced transient stress,” supra, a surface diffusion reflectivity R_{d} of the specimen E is separately measured. The light intensity Φ(0) of the outermost area G just under the surface of the specimen E and the light intensity Φ₀ from the light source 20 which enters the specimen E are given by the following equation.

Φ(0)=(1+7.1R_{d})Φ₀ EQUATION 8

Φ(0) is calculated from Equation 8, and the absorption coefficient μₐ is calculated based on Equation 7 and Φ(0). Next, an effective attenuation coefficient μ_{eff} of the light is calculated by fitting a time profile of the sound pressure in the outermost area G with exp (−μ_{eff}L). The fitting range may be set to a range that corresponds to the outermost area G by converting the time to the distance from the sound velocity. A relationship among the attenuation coefficient μ_{eff}, the absorption coefficient μₐ, and the reduced scattering coefficient μ′ₛ are given as follows:

μ_{eff}=\sqrt{3μₐ(μ′ₛ+μₐ)} EQUATION 9

The reduced scattering coefficient μ′ₛ is calculated from Equation 9, the absorption coefficient μₐ and the attenuation coefficient μ_{eff} which are previously obtained.

Alternatively, the surface diffusion characteristic R_{d}, the absorption coefficient μₐ obtained in Equation 7, and the following equations 10 to 13 that are known with respect to the surface diffusion characteristic R_{d}, the absorption coefficient μₐ and the reduced scattering coefficient μ′ₛ may be used to calculate the reduced scattering coefficient μ′ₛ:

$${R}_{d} = \frac{a}{1 + 2 k ( 1 - a ) + ( 1 + 2 k / 3 ) \sqrt{3 ( 1 - a )}} \qquad EQUATION 10$$

a=μ′ₛ/(μₐ+μ′ₛ) EQUATION 11

$$k = \frac{1 + {r}_{d}}{1 - {r}_{d}} \qquad EQUATION 12$$

r_{d}=−1.44 n⁻²+0.71 n⁻¹+0.0636 n+0.668 EQUATION 13

Here, n is a refractive index of the specimen E.

The step 304 calculates the absorption characteristic (the spectroscopic characteristic) of the outmost area G of the specimen E by using the above method.

The flow from the step 302 to the step 304 is repeated until the outermost area G of the specimen E is measured. After the measurement of the outermost area G ends, the flow moves to the step 305. The step 305 assumes the absorption-scattering characteristic of the measurement site X similarly to the first embodiment. Using this assumption and the known spectroscopic characteristic in the area K, an attenuation amount of the light is estimated from the light incident position to the measurement site X, and the light intensity in the measurement site X is calculated using Equation 6. A predicted value of the sound pressure is calculated using Equation 7.

The step 306 measures a sound pressure of an elastic wave. The step 307 calculates a difference value between the predicted value of the sound pressure obtained in the step 305 and a value of the sound pressure obtained in the step 306, and modifies and calculates the absorption-scattering characteristic of the measurement site X or the measurement area MA as in Equation 5 of the first embodiment. Thus, a local absorption-scattering characteristic can be precisely obtained by recursively calculating the absorption-scattering characteristic of the yet-measured area U with the measured area K.

The above flow is repeated until the step 308 determines that all the measurement areas have been measured. Once all the measurement areas are measured, the image generating unit 13 reads out the results from the memory 14, maps the obtained absorption-scattering characteristic with local positional information, and captures tomographic images of the absorption characteristic and the scattering characteristic of the specimen E. The tomographic images are displayed on the display device 15. The above flow enables the measurement and the calculation of the absorption-scattering characteristic to be performed on a real-time basis, and the result to be displayed on the display device 15.

Even in this embodiment, the image generating unit 13 and the display device 15 can generate an image of functional information such as concentrations of oxygenated hemoglobin, deoxygenated hemoglobin, water, lipid, and collagen, and a hemoglobin metabolism, based on the absorption characteristic obtained with a plurality of wavelengths.

In addition, this embodiment is applicable to a measurement system, like the first and the second embodiments, which puts the specimen E into the measurement vessel 4 having a fixed shape, and fills the matching material 5 between them. This method may be executed concentrically from the outside of the measurement vessel 4 or recursively to the center.

Moreover, like the second embodiment, the memory 14 may consecutively store a measurement result of the entire area of the specimen E and, after the measurement ends, the processing unit 26 may read out the measurement data from the memory 14 and apply this method.

The measurement apparatuses according to the first to third embodiments can precisely and comparatively easily measure the spectroscopic characteristic of the measurement site X in the specimen E (without the reconstruction step disclosed in Japanese Patent No. 3,107,914).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims a foreign priority benefit based on Japanese Patent Application 2007-236711, filed on Sep. 12, 2007, which is hereby incorporated by reference herein in its entirety as if fully set forth herein.

What is claimed is:

1. A measurement apparatus configured to measure a spectroscopic characteristic of a measurement site in a specimen by applying acousto-optical tomography, the measurement apparatus comprising:
a measurement unit configured to measure a light intensity of each of measurement areas that are set differently from the measurement site on a light propagation path from the measurement site to a detection position of a light detector; and
a signal processing device configured to sequentially modify the spectroscopic characteristics of the measurement areas and the measurement site on the light propagation path from the detection position of the light detector to the measurement site by using a light intensity that is measured by the measurement unit in the measurement area that is closer to a surface layer of the specimen than the measurement site.

2. A measurement apparatus according to claim 1, wherein a light incident position upon the specimen and the detection position of the light detector are set such that the light detector can measure light that is incident upon the light incident position and reflected on the measurement area or the measurement site, and
wherein the light incident position upon the specimen and the light detector are configured movable.

3. A measurement apparatus according to claim 1, wherein the signal processing device assumes that two adjacent measurement areas on the light propagation path have the same spectroscopic characteristic,
wherein the signal processing device obtains a difference between an actual measurement value of a light intensity of one of the two adjacent measurement areas which one is closer to the measurement site, and a predicted value of the light intensity of the one measurement area based on a measurement result of the other of the two adjacent measurement areas which is closer to the light detecting device, and
wherein the signal processing device modifies the spectroscopic characteristics of the one measurement area based on a deviation amount corresponding to the difference.

4. A measurement apparatus according to claim 1, further comprising a memory configured to store light intensities of the measurement site and all the measurement areas measured by the measurement unit, before the signal processing device starts processing.

5. A measurement apparatus according to claim 1, wherein the measurement area is set throughout an entire interior of the specimen.

6. A measurement apparatus according to claim 1, wherein the signal processing device forms a three-dimensional tomographic image of the specimen by correlating the spectroscopic characteristic or a concentration and a constituent ratio of an ingredient that contributes to an absorption of the spectroscopic characteristic with a position coordinate of the measurement site or the measurement area,
wherein the measurement apparatus further comprises a display device configured to display the three-dimensional tomographic image.

7. A measurement apparatus configured to measure a spectroscopic characteristic of a measurement site in a specimen by applying photo-acoustic tomography, the measurement apparatus comprising:
a measurement unit configured to measure a photoacoustic signal of each of measurement areas that are set separately from the measurement site on a light propagation path from a light incident position to the measurement site; and
a signal processing device configured to sequentially modify spectroscopic characteristics of the measurement areas and the measurement site on the light propagation path from the light incident position to the measurement site by using a spectroscopic characteristic of a measurement area that is located in an outermost area closest to a surface layer of the specimen measured by the measurement unit.

8. A measurement apparatus according to claim 7, further comprising an ultrasound detecting device configured to detect an ultrasound emitted from the measurement site,
wherein the signal processing device calculates a light intensity of the measurement site based on the spectroscopic characteristic of the measurement area, and calculates the spectroscopic characteristic of the measurement site based on a calculated light intensity of the measurement site and an sound pressure of the ultrasound which is detected by the ultrasound detecting device.

9. A measurement apparatus according to claim 7, wherein the signal processing device assumes that two adjacent measurement areas on the light propagation path have the same spectroscopic characteristic,
wherein the signal processing device obtains a difference between an actual measurement value of an sound pressure of one of the two adjacent measurement areas which one is closer to the measurement site, and a predicted value of the sound pressure of the one measurement area based on a measurement result of the other of the two adjacent measurement areas which is closer to the light incident position, and
wherein the signal processing device modifies the spectroscopic characteristics of the one measurement area based on a deviation amount corresponding to the difference.

10. A measurement apparatus according to claim 7, further comprising a memory configured to store a sound pressure intensity of the measurement site and all the measurement areas measured by the measurement unit, before the signal processing device starts processing.

11. A measurement apparatus according to claim 7, wherein the measurement area is set throughout an entire interior of the specimen.

12. A measurement apparatus according to claim 7, wherein the signal processing device forms a three-dimensional tomographic image of the specimen by correlating the spectroscopic characteristic or a concentration and a constituent ratio of an ingredient that contributes to an absorption of the spectroscopic characteristic with a position coordinate of the measurement site or the measurement area,
wherein the measurement apparatus further comprises a display device configured to display the three-dimensional tomographic image.
